(19) United States
(12) Patent Application Publication
(10) Pub. No.: US 2025/0312510 A1
(43) Pub. Date: Oct. 9, 2025

(54) METHOD OF MAKING A COMPOSITION WITH A FILM-COATED POROUS MATERIAL

(71) Applicant: MEDSKIN SOLUTIONS DR. SUWELACK AG, Billerbeck (DE)

(72) Inventors: Marco WOGRAM, Oststeinbek (DE); Annalena VÖLKER, Nottuln (DE); Diana FERRO, Hamburg (DE)

(73) Assignee: MEDSKIN SOLUTIONS DR. SUWELACK AG, Billerbeck (DE)

(21) Appl. No.: 18/869,692

(22) PCT Filed: Jun. 1, 2023
(86) PCT No.: PCT/EP2023/064692

§ 371 Date: Nov. 26, 2024

(30) Foreign Application Priority Data
Jun. 1, 2022 (EP) 22176711.4

(51) Int. Cl. A61L 15/42; A61L 15/32; A61L 27/24; A61L 27/56; A61L 27/60

(52) U.S. Cl. CPC A61L 15/425; A61L 15/325; A61L 27/24; A61L 27/56; A61L 27/60; A61L 2420/02

(57) ABSTRACT

The present invention relates to a method of making a composition comprising a film-coated porous material and the corresponding composition. The invention further relates to the use of this product in cosmetic skin treatment and treatment of stagnating wounds, split skin graft and ulcers.

### TECHNICAL FIELD

The present invention relates to a method of making a composition comprising a film-coated porous material and the corresponding composition. The invention further relates to the use of this product in cosmetic skin treatment and treatment of stagnating wounds, split skin graft and ulcers.

### BACKGROUND OF THE INVENTION

A variety of coating techniques are employed in order to deposite a material onto a substrate, which include chemical or physical vapor deposition, electrochemical techniques, spraying, slot-die coating, etc. In particular, the use of electrostatic powder coating techniques to coat electrically conductive substrates, such as metals, is well known. By this method, a powder coating material is statically charged and then sprayed or blown onto a surface of a conductive material to which it adheres. The material is impregnated with the powder by means of electrostatic attraction between the positively charged or ionized powder and negatively charged surface of the conductive material or vise verca. This method is particularly used for painting metal articles.

Another well-established technique for the application of solutions onto typically planar substrates is slot-die coating. Slot-die coating allows micron-thick layers to be reliably coated on a flat substrate surface at relatively high speeds, e.g. as indicated in U.S. Pat. No. 7,097,673B2. The coating material is typically dissolved or suspended into a solution or slurry and applied onto a surface of the substrate through a precise coating head known as a slot-die. Slot-die coating is thus a continuous coating technique which delivers quantitatively precise amounts of material, typically of low viscosity, onto a surface at a relatively high speed.

Common usages of slot-die coating technologies are generally limited to smooth nonporous materials, such as photographic films and papers. An important technical issue related to coating porous substrates is how to predict and control the fluid penetration into pores, which directly affects the appearance, properties, and performance of the resulting material. Porous materials are highly attractive as controlled drug carriers because of their large surface area, tunable pore size and mechanical stability. In particular, porous materials are widely used in medical devices as antibacterial, antithrombosis and wound healing agents. However, uncontrolled penetration of a liquid coating containing a healing agent into pores may negatively impact the healing properties of such a device. Therefore, the stability of the liquid coating onto a surface of a porous material is of particular importance for devices for topical administration. For the application in i.e. skin care treatment it is important that the uptake of fluid in terms of time needed to wetten the porouse substrate is as short as possible which is measured by sinking time. Therefore, the sinking time is an important factor in order to maintain porosity of the porouse substrate. Also keeping the pores free from coating is important for medical applications since the pores work as a dermal template wherein cells can grow.

The difference between a slot-die coated (contactless coating, by which the coating follows the surface topologically) and a contact-coating method (such as knife coating or roll, or any type of printing etc) is that the slot-die coating lays an equally distributed film on top of the porous material whereas the contact coating presses the coating into a substrate. Hence the coating blocks and destroys the pores in such a way that the coating comes through the other side.

### DESCRIPTION OF THE INVENTION

In one aspect, the present invention relates to a method of making a composition comprising a porous material, wherein said porous material is essentially flat and comprises a plurality of open and interconnected pores with pore surfaces, comprising the steps of:
a) Providing a porous material, wherein said porous material is essentially flat and comprises a plurality of open and interconnected pores with pore surfaces, wherein the density of said porous material is in the range of from 0.01 to 1 g/cm³ and wherein said pores have an average diameter in the range of from 10 to 150 μm;
b) Providing a coating solution, wherein the viscosity of said coating solution is in the range of from 1 to 20 Pa·s;
c) Providing an applicator means for applying the coating solution onto the surface of said porous material, wherein said applicator means comprises a slot-die;
d) Applying a quantitative amount of the coating solution onto the surface of said porous material to coat said porous material and form a liquid layer by relatively moving said porous material and the applicator means with respect to each other at a speed in the range of from 0.1 to 10 m per minute;
e) Drying said liquid layer.

The method according to the present invention based on a slot-die technique allows to cover the surface of the porous material with a thin layer of a film coating, which predominantly remains on the surface of the porous material and does not penetrate inside the pores. This effect is achieved by balancing the density of the porous material and the viscosity of the applied coating solution. It was found that coating solutions with the viscosities in the range of from 1 to 20 Pa·s can be applied to porous materials with a very low density starting from 0.01 g/cm³. The density of the porous material increases by 10 to 30% after the coating, which contributes to maintaining the coating material on the surface of the substrate. At the same time, the porous structure of the porous material is maintained what is essential for controlled delivery of a healing agent.

Furthermore, the method according to the present invention is contactless so that the applicator means does not contact the surface of the porous material, in particular in step d). It is achieved by positioning the applicator means at a distance to the surface of the porous material. Pores tend to be partially blocked by the coating solution going inside the pores when a contact method is applied. With the contactless method the coating predominantly remains on the surface of the porous material and does not penetrate inside the pores.

In another aspect, the invention relates to the corresponding composition, in particular to the composition obtainable by the method according to the invention.

In yet another aspect, the invention relates to use of a composition comprising a porous material obtainable by the method according to the invention in cosmetic skin treatment, such as treatment or prevention of wrinkles, skin irritation, and as a medicament, in particular for treatment of stagnating wounds, split skin graft and ulcers

### DETAILED DESCRIPTION

In one aspect, the present invention relates to a method of making a composition comprising a porous material, wherein said porous material is essentially flat and comprises a plurality of open and interconnected pores with pore surfaces, comprising the steps of:
a) Providing a porous material, wherein said porous material is essentially flat and comprises a plurality of open and interconnected pores with pore surfaces, wherein the density of said porous material is in the range of from 0.01 to 1 g/cm³, preferably in the range of from 0.02 to 0.05 g/cm³, most preferred in the range of from 0.02 to 0.04 g/cm³ and wherein said pores have an average diameter in the range of from 10 to 150 μm;
b) Providing a coating solution, wherein the viscosity of said coating solution is in the range of from 1 to 20 Pa·s, preferably from 8 to 15 Pa·s, more preferably from 10 to 14 Pa·s;
c) Providing an applicator means for applying the coating solution onto the surface of said porous material, wherein said applicator means comprises a slot-die;
d) Applying a quantitative amount of the coating solution onto the surface of said porous material to coat said porous material and form a liquid layer by relatively moving said porous material and the applicator means with respect to each other at a speed in the range of from 0.1 to 10 m per minute;
e) Drying said liquid layer.

In one embodiment, the density of said porous material increases by 10 to 30%, preferably by 10 to 20% after the coating to form a porous substrate of said composition, in the method of making a composition comprising a porous material.

In one embodiment, the quantitative amount of the coating solution applied onto the surface of said porous material in step d) is in the range of from 0.5 to 200 g/m², preferably in the range of from 10 to 100 g/m², most preferred in the range of from 10 to 30 g/m². In one embodiment, the quantitative amount of the coating solution applied onto the surface of said porous material in step d) is in the range of from 0.5 to 200 g/m². In one embodiment, the quantitative amount of the coating solution applied onto the surface of said porous material in step d) is in the range of from 10 to 100 g/m². In one embodiment, the quantitative amount of the coating solution applied onto the surface of said porous material in step d) is in the range of from 10 to 30 g/m².

In one embodiment the thickness of said porous material is in the range of from 100 to 5000 μm, preferably in the range of from 500 to 3000 μm, more preferably in the range of from 1000 to 2000 μm.

In one embodiment the thickness of the coating after step e) is in the range of from 1 to 300 μm, preferably in the range of from 2 to 50 μm, more preferably in the range of from 5 to 10 μm.

In one embodiment, the coating solution in the method of making a composition comprising a porous material comprises a solvent component and a film forming component. In one embodiment, the solvent component is selected from the group consisting of water and an alcohol, such as ethanol, and mixtures thereof. In one embodiment, the solvent component is water. In a particular embodiment, the content of water in the coating solution is in the range of from 0.5 to 50 wt %, preferably in the range of from 2 to 35 wt %, more preferably in the range of from 3 to 30 wt %. In one embodiment, the film forming component is selected from the group comprising hyaluronic acid (HA), polyacrylate, polyurethane and polysaccharides, such as alginate, nanocellulose or carboxymethylcellulose (CMC), or mixtures thereof. In one embodiment, the film forming component is selected from the group consisting of hyaluronic acid (HA), carboxymethylcellulose (CMC), polyacrylate and polyurethane or mixtures thereof. In one embodiment, the film forming component is a mixture of hyaluronic acid (HA) and carboxymethylcellulose (CMC) and the solvent component is water. In one embodiment the weight ratio of hyaluronic acid (HA) and carboxymethylcellulose (CMC) in water ranges from 1:3 to 3:1, preferably from 1:2 to 2:1, most preferred the weight ratio of hyaluronic acid (HA) and carboxymethylcellulose (CMC) in water is 1:1. In one embodiment the coating solution comprises 0.5 to 3 wt % of hyaluronic acid (HA), 0.5 to 3 wt % of carboxymethylcellulose (CMC) and 94-99 wt % water. In one embodiment the coating solution comprises ca 1 wt % of hyaluronic acid (HA), ca 1 wt % of carboxymethylcellulose (CMC) and ca 98 wt % water.

In one embodiment, the coating solution in the method of making a composition comprising a porous material comprises an emulgator. In one embodiment the emulgator is selected from the group consisting of polyglyceryl-10 laurate, glycerine, sucrose stearate, methyl glucose sequistearate, glyceryl stearate, cetearyl glucoside, hydrogenated palm glycerides, polyethyleneglycole and mixtures thereof.

In one embodiment, the coating solution in the method of making a composition comprising a porous material comprises a further ingredient selected from the group consisting of Vitamin A, Vitamin B, Vitamin C, Vitamin D, Vitamin K, Vitamin E and 4-[(1E,3S)-3-ethenyl-3,7-dimethylocta-1,6-dienyl]phenol (Bakuchiol) or mixtures thereof. Preferably the further ingredient is Vitamin D.

In one embodiment, the porous material in the method of making the composition comprising is in the form of a sheet, dressing or a rolled sheet. A rolled sheet is an advantageous form for continuous operating mode of coating the porous material with the coating solution.

The porous material in the method of making the composition is essentially flat, i.e. the thickness of the porous material throughout the length and the width of the material does not deviate from the average thickness of the porous material by more than ±20%, preferably ±10%. Therefore, the porous substrate in said composition has two major surfaces from the upper and lower sides (see FIG. 23) with an opposite direction to each other extending throughout the length and the width of the substrate. For example, with an average thickness of 1 mm the thickness of the material throughout its length and width remains in the range from 0.8 to 1.2 mm, preferably in the range of from 0.9 to 1.1 mm. With an average thickness of 2 mm the thickness of the essentially flat material throughout its length and width remains in the range of from 1.6 to 2.4 mm, preferably in the range of from 1.8 to 2.2 mm. In one embodiment the porous material and the porous susbstrate is flat.

In one embodiment, the porous material in the method of making the composition comprising a porous material comprises at least 90% by weight biomaterial, preferably the porous material comprises at least 95% by weight biomaterial, more preferably the porous material comprises at least 98% by weight of biomaterial, most preferred the porous material comprises at least 99% by weight biomaterial.

In one embodiment the density of the porous material in the method of making the composition comprising a porous material is in the range of from 0.01 to 1 g/cm³, preferably is in the range of from 0.02 to 0.05 g/cm³, more preferably is in the range of from 0.02 to 0.04 g/cm³, most preferred of from of 0,022 to 0.03 g/cm³.

In one embodiment, the pores in the porous material in the method of making the composition comprising a porous material have an average diameter in the range of from 10 to 150 μm. In one embodiment, the pores in the porous material in the method of making the composition comprising a porous material have an average diameter in the range of from 15 to 65 μm. In one embodiment, the pores in the porous material in the method of making the composition comprising a porous material have an average diameter in the range of from 15 to 40 μm. In one embodiment, the pores in the porous material in the method of making the composition comprising a porous material have an average diameter in the range of from 25 to 100 μm.

In one embodiment, the viscosity of the coating solution in the method of making the composition comprising a porous material is in the range of from 1 to 20 Pa·s. In a preferred embodiment, the viscosity of the coating solution in the method of making the composition comprising a porous material is in the range of from 5 to 20 Pa·s. In a more preferred embodiment, the viscosity of the coating solution in the method of making the composition comprising a porous material is in the range of from 8 to 15 Pa·s. In a yet more preferred embodiment, the viscosity of the coating solution in the method of making the composition comprising a porous material is in the range of from 10 to 14 Pa·s.

In one embodiment the porous material in the method of making the composition comprising a porous material has a density in the range of from 0.01 to 1 g/cm³ and the viscosity of the coating solution is in the range of from 1 to 20 Pa·s. In a preferred embodiment the porous material in the method of making the composition has a density in the range of from 0.02 to 0.05 g/cm³ and the viscosity of the coating solution is in the range of from 8 to 15 Pa·s. In a more preferred embodiment the porous material in the method of making the composition has a density in the range of from 0.02 to 0.04 g/cm³ and the viscosity of the coating solution is in the range of from 10 to 14 Pa·s.

In one embodiment, the porous material in the method of making the composition comprising a porous material is a biomaterial.

In one embodiment, the porous material in the method of making the composition comprising a porous material is selected from the group comprising natural and/or synthetic polymers or mixtures thereof, in particular polysaccharides, glucosaminoglycans, proteins or mixtures thereof.

In one embodiment, the porous material in the method of making the composition comprising a porous material is selected from the group consisting of collagen, alginate, e.g. calcium alginate, hyaluronic acid, cellulose and plant-based proteins, e.g. pie and soy, or a mixture thereof.

In one embodiment, the porous material in the method of making the composition comprising a porous material is collagen. In one embodiment, the porous material in the method of making the composition comprising a porous material is alginate, in particular calcium alginate.

In one embodiment the porous material in the method of making the composition comprising a porous material is a mixture of collagen and calcium alginate. In one embodiment, the porous material comprises collagen by weight in the range of from 80 to 98% and calcium alginate by weight in the range of from 2 to 20%. In a preferred embodiment, the porous material comprises collagen by weight in the range of from 85 to 95% and calcium alginate by weight in the range of from 5 to 15%. In particular, the porous material comprises collagen about 90% by weight and calcium alginate about 10% by weight.

In one embodiment, the collagene in the porous material in the method of making the composition comprising a porous material is animal derived native collagen with a triple helical structure.

In one embodiment the collagen in the porous material in the method of making the composition comprising a porous material is selected from the group comprising 1 type collagen, 3 type collagen, 5 type collagen or a mixture thereof.

In one embodiment of the method of making the composition comprising a porous material the application of the coating solution onto the surface of said porous material is conducted in the way that said porous material is positioned below the fixed slot-die and moves horizontally underneath said slot-die. In one embodiment the speed of moving said porous material is in the range of from 0.1 to 10 m per minute.

In one embodiment of the method of making the composition comprising a porous material the distance between the slot-die and the surface of the porous material is at least 50 μm, preferably at least 100 μm, more preferably at least 200 μm.

In one embodiment of the method of making the composition comprising a porous material the distance between the slot-die and the surface of the porous material is in a range of from 50 to 1000 μm, preferably from 100 to 800 μm, more preferably from 200 to 600 μm.

In one embodiment of the method of making the composition comprising a porous material the solvent component of the coating solution is water and the drying in step e) is conducted in the way that the final content of the water in the composition after drying is in the range of from 5 to 25 wt %, preferably in the range of from 10 to 18 wt %.

In one embodiment of the method of making the composition comprising a porous material the drying in step e) is conducted by supplying hot air to said liquid layer, wherein the temperature of the hot air is in the range from 30 to 100° C., preferably in the range of from 35 to 50° C.

In another aspect, the invention relates to a composition comprising a porous material obtainable by a method according to any of the preceding embodiments.

In third aspect, the invention relates to the composition comprising a porous material obtainable by a method according to any of the preceding embodiments for use as a medicament. In one embodiment, the invention relates to the composition comprising a porous material obtainable by a method according to any of the preceding embodiments for use in the treatment of stagnating wounds, split skin graft and ulcers.

In forth aspect, the invention relates to the composition comprising a porous material obtainable by a method according to any of the preceding embodiments for in cosmetic skin treatment. In one embodiment, the invention relates to the composition comprising a porous material obtainable by a method according to any of the preceding embodiments for use in treatment or prevention of wrinkles and skin irritation.

In fifth aspect, the invention relates to a use of the composition comprising a porous material obtainable by a method according to any of the preceding embodiments in the treatment of stagnating wounds, split skin graft and ulcers.

In sixth aspect, the invention relates to a use of the composition comprising a porous material obtainable by a method according to any of the preceding embodiments in cosmetic skin treatment, such as treatment or prevention of wrinkles and skin irritation.

In seventh aspect, the invention relates to the method of treatment of stagnating wounds, split skin graft and ulcers comprising administering the composition comprising a porous material obtainable by a method according to any of the preceding embodiments to a subject in the need thereof.

In eighth aspect, the invention relates to a composition comprising
a porous substrate comprising a first major surface and a second major surface, wherein said porous substrate is essentially flat and comprises a plurality of open and interconnected pores with pore surfaces extending through the substrate from the first major surface to the second major surface, wherein said pores have an average diameter in the range of from 10 to 150 μm;
a coating on the first major surface and/or on the second major surface, wherein said coating comprises hyaluronic acid (HA), polyacrylate, polyurethane or polysaccharides, such alginate, nanocellulose, or carboxymethylcellulose (CMC), or mixtures thereof.
characterized in that said composition has water absorption in the range of from 10 to 50 g per g of said composition, preferably from 20 to 40 g per g of said composition.

In one embodiment, the thickness of said porous substrate is in the range of from 1 to 5 mm, preferably from 1 to 3 mm, more preferably from 1 to 2 mm and said composition has water absorption in the range of from 20 to 40 g per g of said composition.

In one embodiment said composition has water absorption which does not deviate from the water absorption of the uncoated porous substrate, preferably collagen, by more than ±50%, preferably ±40%, more preferably ±30%, most preferred ±20%.

In a preferred embodiment, said composition has water absorption in the range of from 25 to 35 g per g of said composition.

In one embodiment, the thickness of said porous substrate is in the range of from 1 mm to 2 mm and said composition has water absorption in the range of from 25 to 35 g per g of said composition.

In one embodiment, said coating in the composition is bound to the first major surface and/or on the second major surface of the porous substrate through non-covalent bonds.

In one embodiment, said composition does not comprise a cross-linking agent binding together the porous substrate and the coating.

In some embodiments, the coating on the first major surface and/or on the second major surface of the porous substrate further comprises a water and/or an alcohol, such as ethanol. In some embodiments the coating on the first major surface and/or on the second major surface comprises water.

In a preferred embodiment said coating comprises hyaluronic acid (HA), carboxymethylcellulose (CMC) and water.

In some embodiments the amount of the coating in said composition is in the range of from 0.1 to 10 wt %, preferably from 0.5 to 3 wt % in relation to the weight of the substrate.

In some embodiments, at least 80%, preferably at least 90%, more preferably at least 95%, most preferred at least 99% of said first major surface and/or second major surface is covered by said coating.

In some embodiments, the second major surface of said porous substrate does not comprise said coating. In one embodiment, the coating on the first major surface and/or on the second major surface of the porous substrate further comprises an emulgator. In one embodiment the emulgator is selected from the group consisting of polyglyceryl-10 laurate, glycerine, sucrose stearate, methyl glucose sequistearate, glyceryl stearate, cetearyl glucoside, hydrogenated palm glycerides, polyethyleneglycole and mixtures thereof.

In one embodiment, the coating on the first major surface and/or on the second major surface of the porous substrate further comprises an ingredient selected from the group consisting of Vitamin A, Vitamin B, Vitamin C, Vitamin D, Vitamin K, Vitamin E, and 4-[(1E,3S)-3-ethenyl-3,7-dimethylocta-1,6-dienyl]phenol (Bakuchiol) or mixtures thereof. Preferably the further ingredient is Vitamin D.

In one embodiment, the porous substrate in said composition comprises at least 90% by weight biomaterial, preferably the porous substrate comprises at least 95% by weight biomaterial, more preferably the porous substrate comprises at least 98% by weight of biomaterial, most preferred the porous substrate comprises at least 99% by weight biomaterial.

In one embodiment, the pores in the porous substrate of said composition have an average diameter in the range of from 10 to 150 μm. In one embodiment, the pores in the porous substrate of said composition have an average diameter in the range of from 15 to 65 μm. In one embodiment, the pores in the porous substrate of said composition have an average diameter in the range of from 15 to 40 μm. In one embodiment, the pores in the porous substrate of said composition have an average diameter in the range of from 25 to 100 μm.

In one embodiment the porous substrate in said composition is a biomaterial.

In one embodiment, the porous substrate in said composition is selected from the group comprising natural and/or synthetic polymers or mixtures thereof, in particular polysaccharides, glucosaminoglycans, proteins or mixtures thereof.

In one embodiment, the porous substrate in said composition is selected from the group consisting of collagen, alginate, e.g. calcium alginate, hyaluronic acid, cellulose and plant-based proteins, e.g. pie and soy, or a mixture thereof.

In one embodiment, the porous substrate in said composition is collagen. In one embodiment, the porous substrate in said composition is alginate, in particular calcium alginate.

In one embodiment the porous substrate in said composition is a mixture of collagen and calcium alginate. In one embodiment, the porous substrate comprises collagen by weight in the range of from 80 to 98% and calcium alginate by weight in the range of from 2 to 20%. In a preferred embodiment, the porous substrate comprises collagen by weight in the range of from 85 to 95% and calcium alginate by weight in the range of from 5 to 15%. In particular, the porous substrate comprises collagen about 90% by weight and calcium alginate about 10% by weight.

In one embodiment, the collagene in the porous substrate is animal derived native collagen with a triple helical structure.

In one embodiment the collagen in the porous substrate is selected from the group comprising 1 type collagen, 3 type collagen, 5 type collagen or a mixture thereof.

In some emdobiments, the methods according to any of the above embodiments are used to prepare said composition as defined in any of the preceding embodiments.

In ninth aspect, the invention relates to the composition according to any of the preceding embodiments for in cosmetic skin treatment. In one embodiment, the invention relates to the the composition according to any of the preceding embodiments for use in treatment or prevention of wrinkles and skin irritation.

In tenth aspect, the invention relates to a use of the composition according to any of the preceding embodiments in the treatment of stagnating wounds, split skin graft and ulcers.

In eleventh aspect, the invention relates to a use of the composition according to any of the preceding embodiments in cosmetic skin treatment, such as treatment or prevention of wrinkles and skin irritation.

In twelveth aspect, the invention relates to the method of treatment of stagnating wounds, split skin graft and ulcers comprising administering the composition according to any of the preceding embodiments to a subject in the need thereof.

With the above context, the following consecutively numbered embodiments provide further specific aspects of the invention:
1. A method of making a composition comprising a porous material, wherein said porous material is essentially flat and comprises a plurality of open and interconnected pores with pore surfaces, comprising the steps of:
   a) Providing a porous material, wherein said porous material is essentially flat and comprises a plurality of open and interconnected pores with pore surfaces, wherein the density of said porous material is in the range of from 0.01 to 1 g/cm³, preferably in the range of from 0.02 to 0.05 g/cm³, most preferred in the range of from 0.02 to 0.04 g/cm³ and wherein said pores have an average diameter in the range of from 10 to 150 μm;
   b) Providing a coating solution, wherein the viscosity of said coating solution is in the range of from 1 to 20 Pa·s, preferably from 5 to 20 Pa·s, more preferably from 8 to 15 Pa·s, even more preferably from 10 to 14 Pa·s;
   c) Providing an applicator means for applying the coating solution onto the surface of said porous material, wherein said applicator means comprises a slot-die;
   d) Applying a quantitative amount of the coating solution onto the surface of said porous material to coat said porous material and form a liquid layer by relatively moving said porous material and the applicator means with respect to each other at a speed in the range of from 0.1 to 10 m per minute;
   e) Drying said liquid layer.
2. The method of making a composition comprising a porous material according to embodiment 1, wherein the density of said porous material increases by 10 to 30%, preferably by 10 to 20% after the coating.
3. The method of making a composition comprising a porous material according to embodiment 1 or 2, wherein the quantitative amount of the coating solution applied onto the surface of said porous material in step d) is in the range of from 0.5 to 200 g/m², preferably in the range of from 10 to 100 g/m², most preferred in the range of from 10 to 30 g/m².
4. The method of making a composition comprising a porous material according to any of embodiments 1 to 3, wherein said coating solution comprises a solvent component and a film forming component.
5. The method of making a composition comprising a porous material according to embodiment 4, wherein said solvent component is water.
6. The method of making a composition comprising a porous material according to embodiment 5, wherein the content of water in said coating solution is in the range of from 0.5 to 50 wt %, preferably in the range of from 2 to 35 wt %, more preferably in the range of from 3 to 30 wt %.
7. The method of making a composition comprising a porous material according to any of embodiments 4 to 6, wherein said film forming component is selected from the group consisting of hyaluronic acid (HA), polyacrylate, polyurethane and polysaccharides, such as alginate, nanocellulose or carboxymethylcellulose (CMC), or mixtures thereof.
8. The method of making a composition comprising a porous material according to any of embodiments 1 to 7, wherein said coating solution comprises an emulsifier.
9. The method of making a composition comprising a porous material according to embodiment 8, wherein said emulsifier is selected from the group consisting of polyglyceryl-10 laurate, glycerine, sucrose stearate, methyl glucose sequistearate, glyceryl stearate, cetearyl glucoside, hydrogenated palm glycerides, polyethyleneglycole or mixtures thereof.
10. The method of making a composition comprising a porous material according to any of embodiments 1 to 9, wherein said coating solution comprises a further ingredient selected from the group consisting of Vitamin A, Vitamin B, Vitamin C, Vitamin K, Vitamin E and 4-[(1E,3S)-3-ethenyl-3,7-dimethylocta-1,6-dienyl]phenol (Bakuchiol), or mixtures thereof.
11. The method of making a composition comprising a porous material according to any of embodiments 1 to 10, wherein said porous material is a biomaterial.
12. The method of making a composition comprising a porous material according to embodiment 11, wherein said biomaterial is selected from the group comprising natural and/or synthetic polymers or mixtures thereof, in particular polysaccharides, glucosaminoglycans, proteins and/or synthetic polymers or mixtures thereof.
13. The method of making a composition comprising a porous material according to embodiment 11 or 12, wherein said biomaterial is selected from the group consisting of collagen, alginate, hyaluronic acid, cellulose, and plant-based proteins such as pie and soy.
14. The method of making a composition comprising a porous material according to embodiment 13, wherein said biomaterial is collagen.
15. The method of making a composition comprising a porous material according to embodiment 14, wherein said biomaterial is animal derived native collagen with a triple helical structure.
16. The method of making a composition comprising a porous material according to any of embodiments 1 to 15, wherein the application of the coating solution onto the surface of said porous material is conducted in the way that said porous material is positioned below the fixed slot-die and moves horizontally underneath said slot-die.
17. The method of making a composition comprising a porous material according to any of embodiments 1 to 16, wherein the distance between the slot-die and the surface of the porous material is in a range of from 50 to 1000 μm, preferably from 100 to 800 μm, more preferably 200 to 600 μm.
18. The method of making a composition comprising a porous material according to any of embodiments 1 to 17, wherein the drying in step e) is conducted by supplying hot air to said liquid layer, wherein the temperature of the hot air is in the range from 30 to 100° C., preferably in the range of from 35 to 50° C.
19. The method of making a composition comprising a porous material according to any of embodiments 1 to 18, wherein said porous material is in the form of a sheet, a dressing, or a rolled sheet.
20. A composition comprising
   a porous substrate comprising a first major surface and a second major surface, wherein said porous substrate is essentially flat and comprises a plurality of open and interconnected pores with pore surfaces extending through the porous substrate from the first major surface to the second major surface, wherein said pores have an average diameter in the range of from 10 to 150 μm;
   a coating on the first major surface and/or on the second major surface of said porous substrate, wherein said coating comprises hyaluronic acid (HA), polyacrylate, polyurethane or polysaccharides, such as alginate, nanocellulose, or carboxymethylcellulose (CMC), or mixtures thereof; characterized in that said composition has water absorption in the range of from 10 to 50 g per g of said composition, preferably from 20 to 40 g per g of said composition.
21. The composition according to embodiment 20, wherein said coating in the composition is bound to the first major surface and/or on the second major surface of said porous substrate through non-covalent bonds.
22. The composition according to embodiment 20 or 21, wherein said composition does not comprise a cross-linking agent binding together the porous substrate and the coating.
23. The composition according to any of embodiments 20 to 22, wherein the coating on the first major surface and/or on the second major surface further comprises water.
24. The composition according to any of embodiments 20 to 23, wherein the amount of coating in said composition is in the range of from 0.1 to 10 wt %, preferably from 0.5 to 3 wt % in relation to the weight of said composition.
25. The composition according to any of embodiments 20 to 24, wherein at least 80%, preferably at least 90%, more preferably at least 95%, most preferred at least 99% of said first major surface and/or second major surface is covered by said coating.
26. The composition according to any of embodiments 20 to 25, wherein the coating on the first major surface and/or on the second major surface of said porous substrate further comprises an emulgator.
27. The composition according to any of embodiments 20 to 26, wherein the coating the coating on the first major surface and/or on the second major surface of said porous substrate further comprises an ingredient selected from the group consisting of Vitamin A, Vitamin B, Vitamin C, Vitamin D, Vitamin K, Vitamin E and 4-[(1E,3S)-3-ethenyl-3,7-dimethylocta-1,6-dienyl]phenol (Bakuchiol) or mixtures thereof.
28. The composition according to any of embodiments 20 to 27, wherein the porous substrate in said composition comprises at least 90% by weight biomaterial, preferably at least 95% by weight biomaterial, more preferably at least 98% by weight of biomaterial, most preferred at least 99% by weight biomaterial.
29. The composition according to any of embodiments 20 to 28, wherein the porous substrate in said composition is a biomaterial.
30. The composition according to any of embodiments 20 to 29, wherein the porous substrate in said composition is selected from the group comprising natural and/or synthetic polymers or mixtures thereof, in particular polysaccharides, glucosaminoglycans, proteins or mixtures thereof.
31. The composition according to any of embodiments 20 to 30, wherein the porous substrate in said composition is selected from the group consisting of collagen, alginate, e.g. calcium alginate, hyaluronic acid, cellulose and plant-based proteins, e.g. pie and soy, or a mixture thereof.
32. The composition according to embodiments 31, wherein the porous substrate in said composition is collagen.
33. The composition according to embodiment 31 or 32, wherein the porous substrate in said composition is a mixture of collagen and calcium alginate.
34. The composition according to any of embodiments 31 to 33, wherein the collagene in the porous substrate is animal derived native collagen with a triple helical structure.
35. The composition according to any of embodiments 31 to 34, wherein the collagene in the porous substrate is is selected from the group comprising 1 type collagen, 3 type collagen, 5 type collagen or a mixture thereof.
36. A composition obtainable by a method according to any of embodiments 1 to 19.
37. The composition according to any of embodiments 20 to 36 for use as a medicament.
38. The composition according to any of embodiments 20 to 36 for use in the treatment of stagnating wounds, split skin graft and ulcers.
39. The composition according to any of embodiments 20 to 36 for use in cosmetic skin treatment, such as treatment or prevention of wrinkles and skin irritation.
40. Use of the composition according to any of embodiments 20 to 36 in the treatment of stagnating wounds, split skin graft and ulcers.
41. Use of the composition according to any of embodiments 20 to 36 in cosmetic skin treatment, such as treatment or prevention of wrinkles and skin irritation.
42. Method of treatment of stagnating wounds, split skin graft and ulcers comprising administering the composition according to any of embodiments 20 to 36 to a subject in the need thereof.

### Definitions

Listed below are definitions of various terms used to describe this invention. These definitions apply to the terms as they are used throughout this specification and claims unless otherwise limited in specific instances either individually or as part of a larger group. Unless defined otherwise all technical and scientific terms used herein generally have the same meaning as commonly understood by one of ordinary skill in the art to which this invention belongs.

As used herein, the articles “a” and “an” refer to one or to more than one (i.e. to at least one) of the grammatical object of the article. By way of example, “an element” means at least one element, i.e. an element or more than one element.

As used herein, the term “porous material” refers to a material comprising pores, i.e. cavities, channels or interstices, wherein the depth of the pores exceeds their average diameter.

As used herein, the term “porous substrate” refers to the porous material of said composition on which the coating is applied.

Unless specifically defined, the term “thickness” related to a porous material refers to an average thickness of the porous material.

As used herein, the term “essentially flat” refers to the material with the thickness throughout the length and the width not deviating from the average thickness by more than +20%, preferably +10%.

As used herein, the term “sheet” refers to an essentially flat material, wherein the thickness of the material is in the range of from 1 to 8 mm.

As used herein, the terms “rolled material” and “rolled sheet” are used interchangeably and refer to the sheet of porous material wound cylindrically about a center axis of a roll with the possibility of removal of the material from the center or inner periphery of the roll.

As used herein, the term “dressing” refers to the composition comprising a film-coated porous material, which is further coated with an additional layer of a polymer, wherein said composition is in a form of a sheet.

As used herein the term “applicator means” refers to the means suitable for applying a liquid onto a surface of a material.

As used herein, the term “solvent component” refers to a liquid comprised of a single solvent or a mixture of solvents, which is suitable for solubilizing or dispersing one or more of a variety of substances. Non-limiting examples of such solvents include water and alcohols, such as ethanol or isopropanol.

As used herein, the term “film forming component” refers to a substance capable of forming a film upon application to a solid surface. In particular, the film forming component is applied to a surface in a form of a liquid layer and film is formed after the liquid layer dries in air. The non-limiting examples of film forming components include hyaluronic acid (HA), carboxymethyl cellulose (CMC), nanocellulose, alginate, polyacrylate and polyurethane.

As used herein, the term “emulsifier” refers to a substance, which facilitates mixing of immiscible liquids with different polarities, e.g. water with lipophilic ingredients. Non-limiting examples of emulsifiers include polyglyceryl-10 laurate, polyglyceryl-10 laurate, glycerine, sucrose stearate, methyl glucose sequistearate, glyceryl stearate, cetearyl glucoside, hydrogenated palm glycerides, polyethyleneglycole.

As used herein, the term “coating” refers to a thin deposit of a material that substantially covers the surface of a substrate.

As used herein, the term “salt” refers to ionic a chemical compound consisting of an ionic assembly of a positively charged cation and a negatively charged anion.

As used herein, the term “biomaterial” refers to a natural or synthetic biocompatible material which is suitable for using in a medical device, intended to interact with biological systems. Non-limiting examples include collagen, gelatine, alginate and polysaccharides such as glycosaminoglycans.

As used herein, the term “polysaccharide” refers to polymers comprising a backbone comprised mainly of (at least 90%) monosaccharide repeating units and/or derivatized monosaccharide repeating units.

As used herein, the term “protein” or “polypeptide” refers to a polymer of two or more of the natural amino acids or non-natural amino acids.

As used herein the term “glycosaminoglycan” refers to a group of acid polysaccharides, each having a repeating unit of disaccharide consisting of an amino sugar and uronic acid or galactose.

As used herein, the term “alginate” refers to the anion of alginic acid. Therefore, the terms “alginate” and “alginate salt” are used interchangeable in the context of the present invention. The alginate salt can be, for example, calcium alginate. Alginate is a linear polymer formed by anions of β-D-mannuronic acid (M, β-D-mannuronate) and of α-L-guluronic acid (G, α-L-guluronate) bound by means of 1-4 glycosidic bonds.

As used herein, the term “collagen” refers to the extracellular family of fibrous proteins that are characterised by their stiff, triple-stranded helical structure. Three collagen polypeptide chains (“α-chains”) are wound around each other to form this helical molecule.

As used herein the term “subject” refers to a human or a non-human mammal. Preferably the subject is human.

### DESCRIPTION OF FIGURES

FIG. 1 shows a schematic view of a slot-die coating process.

FIG. 2 shows a SEM (scanning electron microscope) image of a collagen matrix without coating, top view.

FIG. 3 shows a SEM (scanning electron microscope) image of a collagen matrix with CMC coating, top view.

FIG. 4 shows a SEM (scanning electron microscope) image of a collagen matrix with 1% HA coating (20 μm) applied by a contactless slot-die method (Example 2, Experiment 1), top view

FIG. 5 shows a SEM (scanning electron microscope) image of a collagen matrix with 1% HA coating (20 μm) applied by a contactless slot-die method (Example 2, Experiment 1), cross-section (150× magnification)

FIG. 6 shows a SEM (scanning electron microscope) image of a collagen matrix with 1% HA coating (20 μm) applied by a contactless slot-die method (Example 2, Experiment 1), cross-section (1500× magnification)

FIG. 7 shows a SEM (scanning electron microscope) image of a collagen matrix with 1% HA coating (20 μm) applied by a contactless slot-die method (Example 2, Experiment 1), bottom view

FIG. 8 shows a SEM (scanning electron microscope) image of a collagen matrix with 1% HA coating (40 μm) applied by a contactless slot-die method (Example 2, Experiment 2), top view FIG. 9 shows a SEM (scanning electron microscope) image of a collagen matrix with 1% HA coating (40 μm) applied by a contactless slot-die method (Example 2, Experiment 2), cross-section (150× magnification)

FIG. 10 shows a SEM (scanning electron microscope) image of a collagen matrix with 1% HA coating (40 μm) applied by a contactless slot-die method (Example 2, Experiment 2), cross-section (1500× magnification)

FIG. 11 shows a SEM (scanning electron microscope) image of a collagen matrix with 1% HA coating (40 μm) applied by a contactless slot-die method (Example 2, Experiment 2), bottom view

FIG. 12 shows a SEM (scanning electron microscope) image of a collagen matrix with 1% HA coating (30 μm) applied by a contact method (Example 2, Experiment 3), top view

FIG. 13 shows a SEM (scanning electron microscope) image of a collagen matrix with 1% HA coating (30 μm) applied by a contact method (Example 2, Experiment 3), cross-section (150× magnification)

FIG. 14 shows a SEM (scanning electron microscope) image of a collagen matrix with 1% HA coating (30 μm) applied by a contact method (Example 2, Experiment 3), cross-section (1500× magnification)

FIG. 15 shows a SEM (scanning electron microscope) image of a collagen matrix with 1% HA coating (30 μm) applied by a contact method (Example 2, Experiment 3), bottom view

FIG. 16 shows a SEM (scanning electron microscope) image of a collagen matrix without coating, cross-section (150× magnification)

FIG. 17 shows a SEM (scanning electron microscope) image of a collagen matrix without coating, cross-section (1500× magnification) FIG. 18 shows a SEM (scanning electron microscope) image of a collagen matrix with 0.4% HA coating (40 μm) applied by a contactless slot-die method (Example 3), top view

FIG. 19 shows a SEM (scanning electron microscope) image of a collagen matrix with 0.4% HA coating (40 μm) applied by a contactless slot-die method (Example 3), cross-section (100× magnification)

FIG. 20 shows a SEM (scanning electron microscope) image of a collagen matrix with 0.4% HA coating (40 μm) applied by a contactless slot-die method (Example 3), cross-section from the upper side (1500× magnification)

FIG. 21 shows a SEM (scanning electron microscope) image of a collagen matrix with 0.4% HA coating (40 μm) applied by a contactless slot-die method (Example 3), cross-section to the lower side (1500× magnification)

FIG. 22 shows a SEM (scanning electron microscope) image of a collagen matrix with 0.4% HA coating (40 μm) applied by a contactless slot-die method (Example 3), bottom view

FIG. 23 shows the orientation of two major surfaces of an uncoated collagen matrix

### EXAMPLES

### Example 1

### Coating Solution 1

CMC (Carboxymethylcellulose): 3.3 wt %
Water: 96.7 wt %.

### Coating Solution 2

Vitamin C: 25 wt %,
CMC (Carboxymethylcellulose): 3.3 wt %,
Water: 71.7 wt %

### Coating Solution 3

4-[(1E,3S)-3-ethenyl-3,7-dimethylocta-1,6-dienyl]phenol (Bakuchiol): 5 wt %
polyglyceryl-10 laurate: 5 wt %,
CMC (Carboxymethylcellulose): 4 wt %,
Water: 86 wt %

### Coating Procedure:

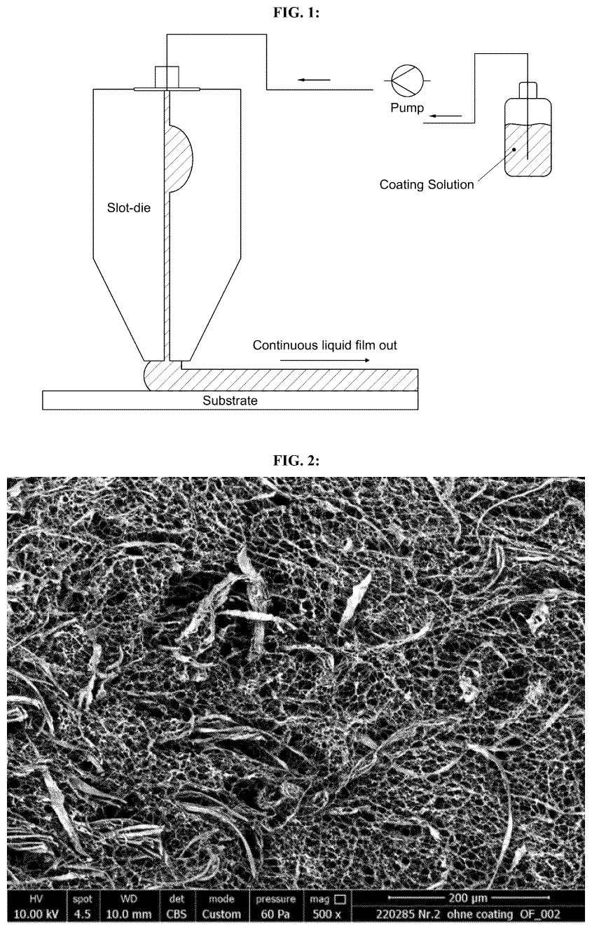

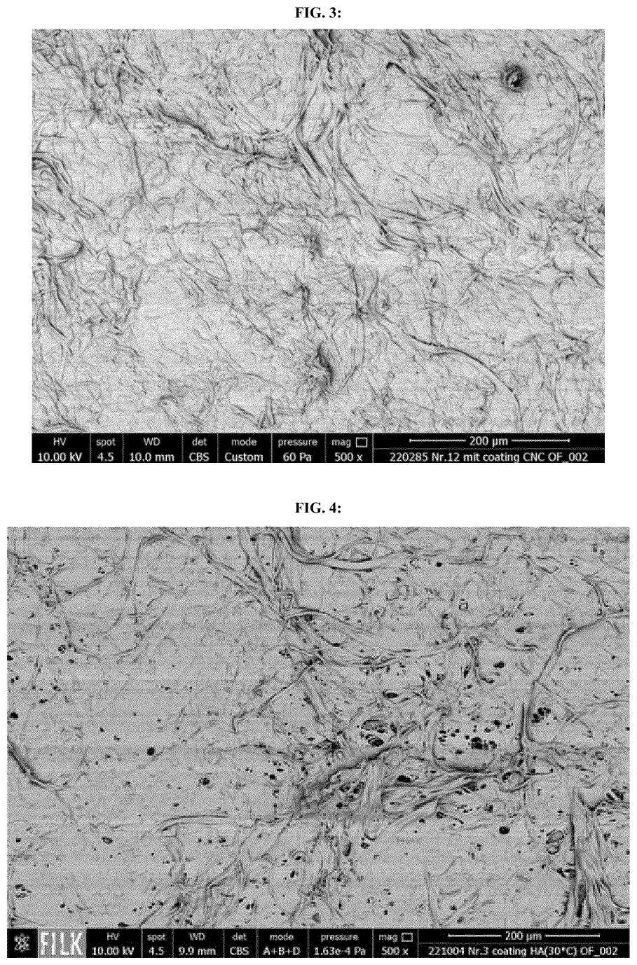

The coating was applied by means of a slot-die process. A schematic view of the process is depicted in FIG. 1. The sequence of the process steps is as follows:
A porous collagen matrix substrate with the dimensions of 46×33×0.15 cm and the density of 0,022 g/cm³ is placed on a movable table
A slot-die is positioned at a distance of 700 μm from the plane of the substrate
The table is arranged to move horizontally underneath the slot-die with the speed of 1 m/min.
When the slot-die reaches the position above the substrate, the pump is arranged to pump the coating solution with the flow rate 5 ml/min
After the coating is evenly distributed across the substrate, the pump is switched off.
The applied coating is dried in an oven at 90° C. for 2 minutes so that a thin coating film on the substrate is formed. The SEM images of the uncoated collagen matrix and the collagen matrix coated with CMC (Coating solution 1) are provided in FIGS. 2 and 3, respectively.

### Example 2

A series of comparative experiments have been conducted to demonstrate the advantageous properties of the product, which is prepared by the method according to the invention. The results are summarized in Table 1.

A porous collagen matrix has been coated by means of a contactless slot-die process by applying 1.1% HA coating aqueous solution with a viscosity of 11.8 Pa·S (measured by a viscometer) with 20 μm (Experiment 1) and 40 μm (Experiment 2) thickness as described in Procedure 2a below:

### Procedure 2a

A porous collagen matrix substrate with the dimensions of 460 mm×330 mm×1.5 mm and the density of 0,024 g/cm′ is placed on a movable table
A slot-die is positioned at a distance of 350 μm (for 20 μm) and 700 μm (for 40 μm) from the plane of the substrate.
The table is arranged to move horizontally underneath the slot-die with the speed of 1 m/min
When the slot-die reaches the position above the substrate, the pump is arranged to pump 1.1% HA coating aqueous solution with the flow rate 6.3 ml/min to form a 20.1 μm layer (Experiment 1) or the flow rate 12.6 ml/min to form a 40.1 μm layer (Experiment 2)
After the coating is evenly distributed across the substrate, the pump is switched off.
The applied coating is dried in an oven at 50° C. for 2 minutes so that a thin coating film on the substrate is formed.

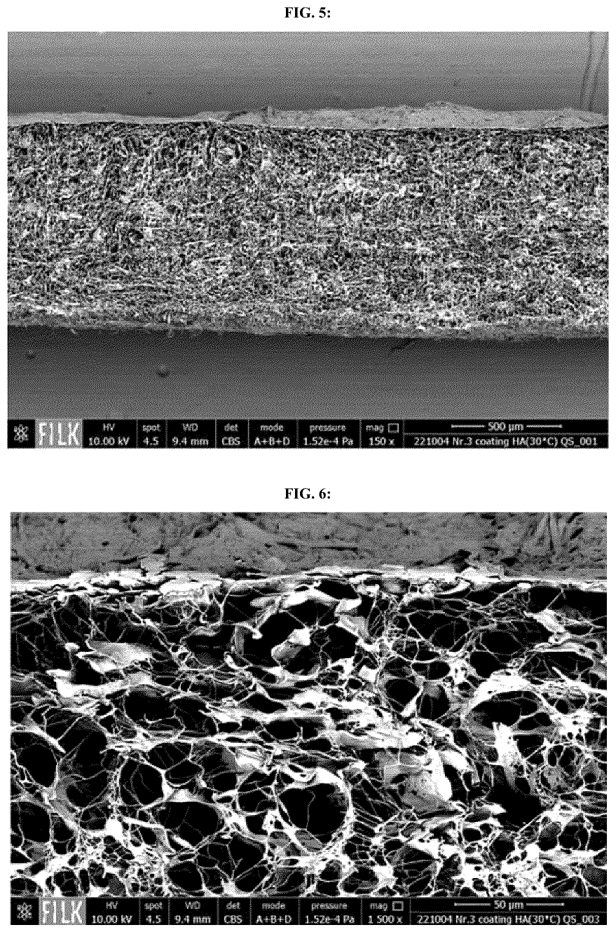

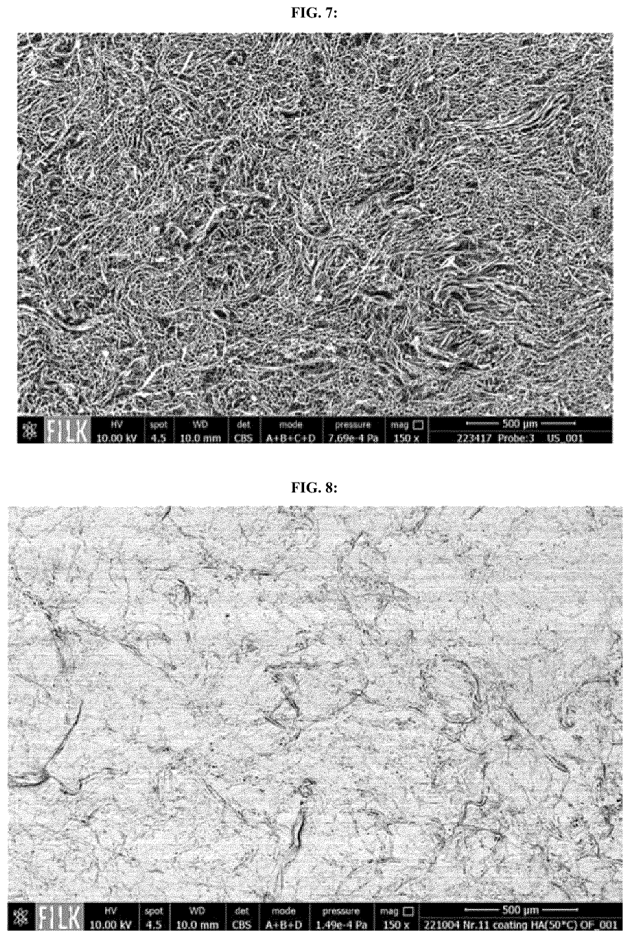

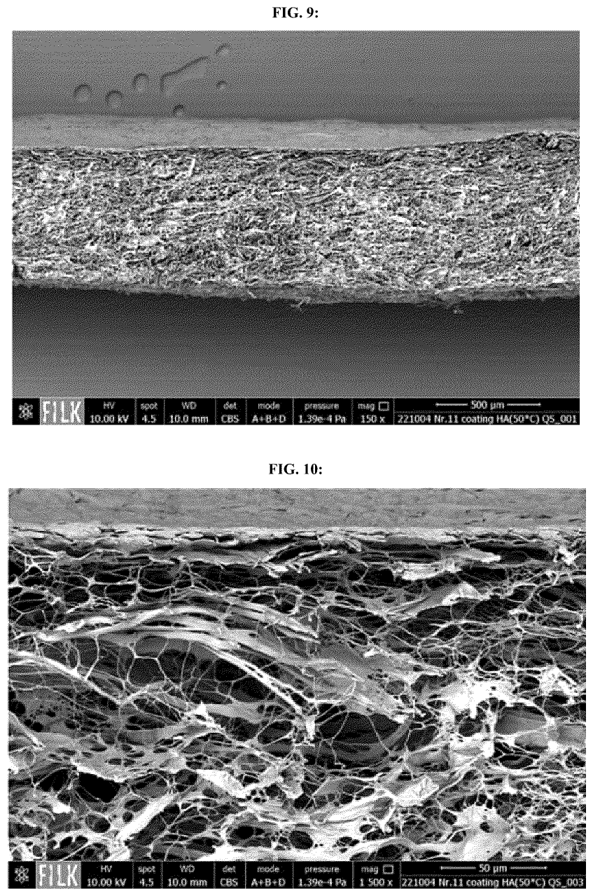

The SEM images of the obtained materials are depicted as FIGS. 4-7 (Experiment 1) and 8-11 (Experiment 2).

As a comparison, a porous collagen matrix has been coated by means of a contact process by applying 1.1% HA coating aqueous solution with 30 μm (Experiment 3) thickness as described in Procedure 2b below:

### Procedure 2b

A knife hand coater was placed onto the collagen whereas the knife has a distance of 30 μm (a rectangular shape where 30 μm are taken away by i.e. CNC grinding) from the surface 21ompar collagen sheet.

Then the coating is placed behind the knife and the knife is pulled over the surface 22ompar collagen sheet. The excess amount at the end of the coating length is then taken off with a wipe of the surface (this part was not taken into any measurement or consideration). The sample was dried in a convectional oven for 2 min at 50° C.

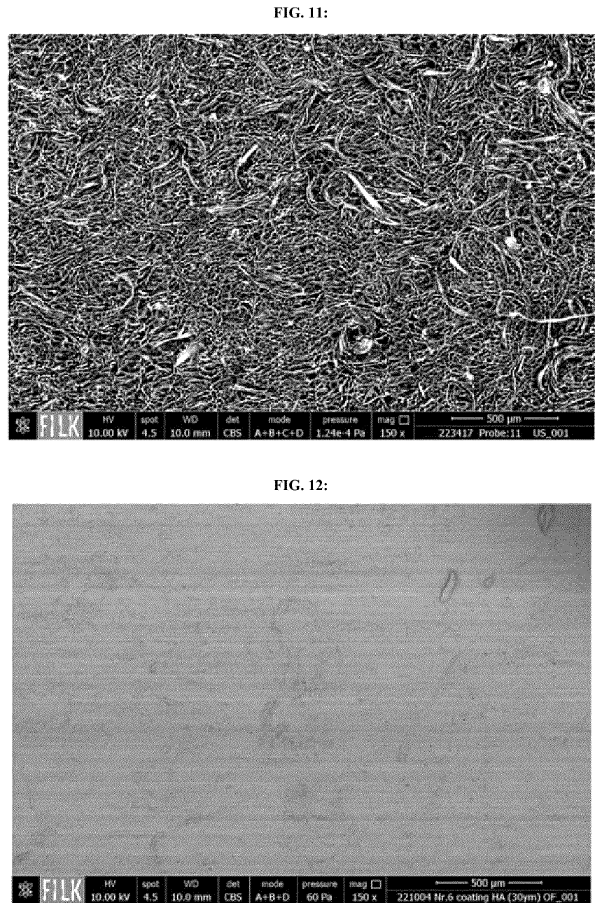

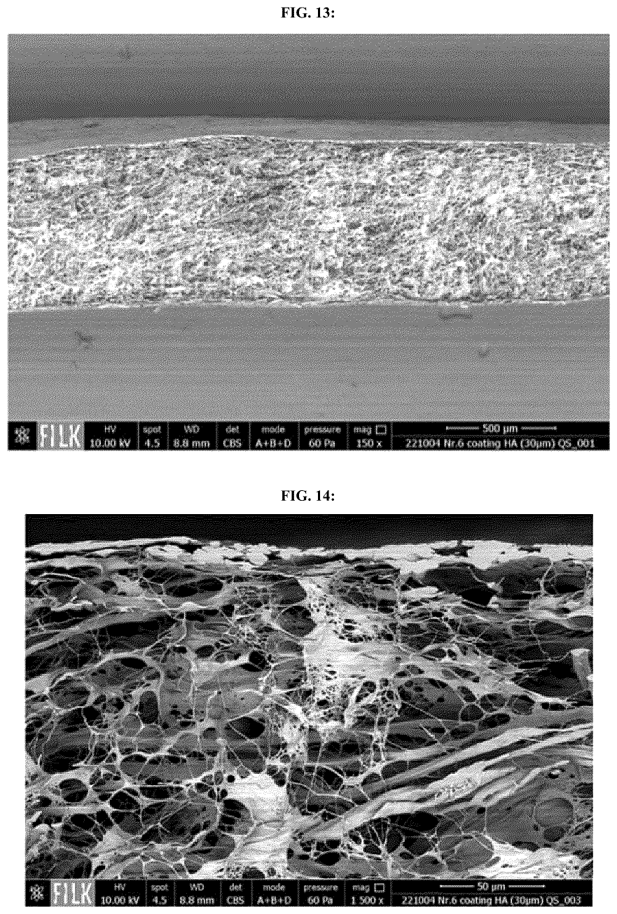

The SEM images of the obtained material are depicted as FIGS. 12-15.

The sinking time in water as well as water absorption of the obtained materials have been measured (summarized results are in Table 1) as described in Procedure 2c below:

### Procedure 2c:

A wired basket is weighed and the weight is recorded (m₁)
The collagen sample is weighed, the weight is recorded and the sample is plugged in the wired basket
The wired basket with the sample is weighed and the weight is recorded (m₂)
A 1000 ml beaker is filled with RO water, wherein the temperature of water is controlled
The beaker with water is placed on a scale and tared
The wired basked with the sample is dropped horizontally into a beaker from the height of ca 1 cm above the surface of water
At the same time recording of time begins, which is required for the wired basked with the sample to sink completely under the surface of water
The wired basked with the sample is taken out from water and is kept for ca 30 seconds above water to allow the sample to drain
The wired basket with the sample is weighed and the weight is recorded (m₃)

The water absorption is calculated as A=(m₃−m₂)/(m₂−m₁).

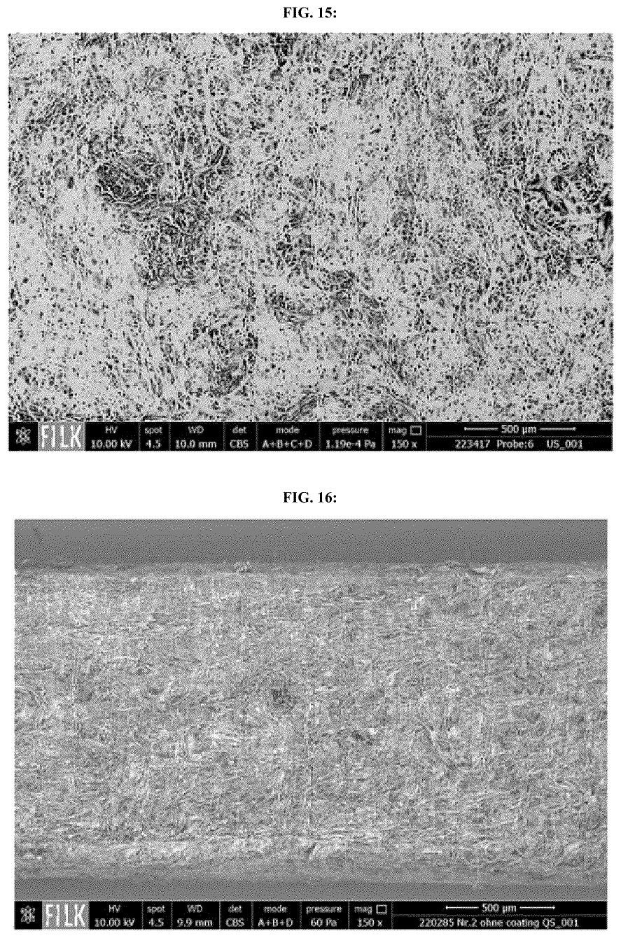

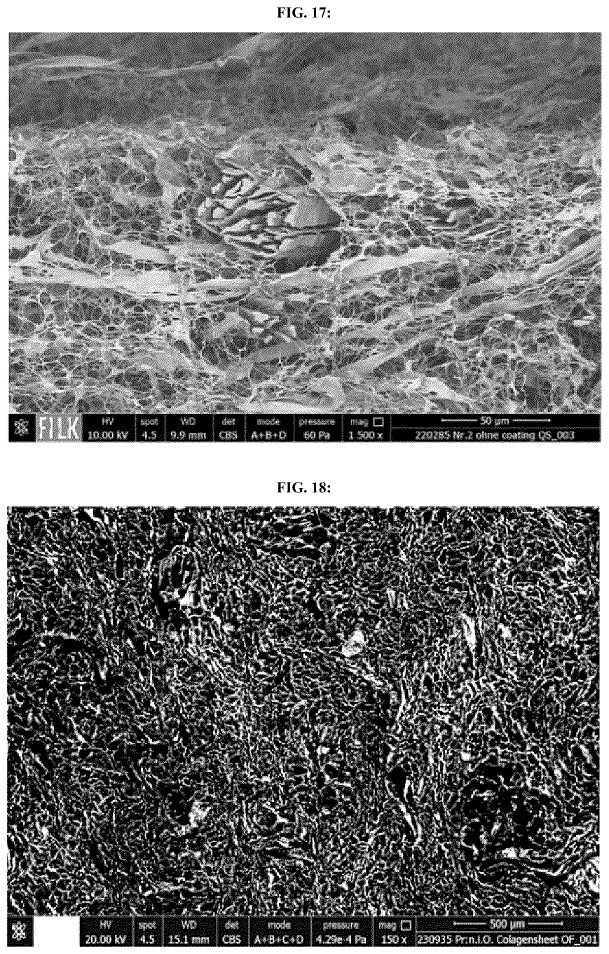

As a reference, the same measurements have been conducted with an uncoated collagen matrix (Experiment 4, SEM images are depicted as FIGS. 2, 16 and 17) as well as milled and dried collagen (prepared by dispersing of 2% of finely grounded collagen in water by intense mixing, pouring the slurry onto a tea paper, vacuuming the slurry through the tea paper to remove most of water and drying the remaining slurry in a convection oven till the residue moisture of 5 to 12% is achieved) (Experiment 5) and printing paper (Example 6).

TABLE 1
The results of measurements of sinking time and water absorption

| | | Sinking | Water absorption |
|---|---|---|---|
| Experiment | Measurement | time-s | in g/g |
| 1 (slot-die, 20 μm | 1 | 34 | 28.19 |
| coating) | 2 | 27 | 27.58 |
| 2 (slot-die, 40 μm | 1 | 49 | 26.4 |
| coating) | 2 | 38 | 27.1 |
| 3 (contact, 30 μm | 1 | 64 | 27.96 |
| coating) | 2 | 62 | 27.38 |
| 4 (uncoated collagen) | 1 | 18 | 32.93 |
| | 2 | 35 | 32.76 |
| 5 (milled and | 1 | 3 | 3.60 |
| compressed collagen) | 2 | 6 | 3.77 |
| 6 (printing paper) | 1 | Not observed | 1.66 |
| | 2 | Not observed | 1.83 |

The purpose of Experiment 5 was to simulate the compressed collagen foam film from U.S. Pat. No. 3,800,792 A. As can be seen from Table 1, the printing paper as well as milled and compressed collagen film both have very poor water permeability and absorbancy even when uncoated. A further drop of values can be obviously expected if a coating is applied to said materials. In contrast, the coated porous collagen prepared by the slot-die method according to the present invention (Experiments 1 and 2) as well as comparative experiment with a contact method (Experiment 3) has water absorbancy which is comparable to those of the uncoated porous collagen. However, the material prepared by the contactless slot-die method according to the present invention has a further advantage of a lower sinking time in water compared to the material produced with a contact method. For example, water needs around 40 seconds to go through the material obtained in Experiment 2 compared to ca 60 seconds for the material obtained in Experiment 3, although a thicker coating is applied in the first case. This can be explained by pores being partially blocked by the coating solution going inside the pores when a contact method is applied. When coating runs through the porous substrate, it takes a longer time to dissolve it, which results in an increased sinking time. So, the porous collagen coated by s slot-die contactless method wettens more rapidly, which has advantages in particular in skin treatment.

This effect can be also seen from the SEM images. The top view of the coated collagen materials obtained by a contactless slot-die method (FIGS. 4 and 8) reveals clearly seen pores and cavities, whereas almost no pores can be seen when a contact method is applied (FIG. 12). The difference can be also observed when bottom sides of the respective materials are compared. While the bottom view of the materials prepared by a contactless slot-die method reveals no substantial difference compared to the uncoated porous collagen (FIGS. 7, 11 and 2), the bottom side of the material obtained by a contact method clearly indicates the reduced porosity.

### Example 3

A comparative experiment with a slot-die coating solution with a viscosity of 3 Pa·s has been conducted, as described in the procedure below:

A porous collagen matrix has been coated by means of a contactless slot-die process by applying 0.4%

HA coating aqueous solution with 40 μm at a viscosity of 3 Pa·s (measured by a viscometer).
A porous collagen matrix substrate with the dimensions of 460 mm×330 mm×1.5 mm and the density of 0,024 g/cm³ is placed on a movable table
A slot-die is positioned at a distance of 600 μm from the plane of the substrate.
The table is arranged to move horizontally underneath the slot-die with the speed of 1 m/min.
When the slot-die reaches the position above the substrate, the pump is arranged to pump 0.4% HA coating aqueous solution with the flow rate 12 ml/min to form a 40 μm layer
After the coating is evenly distributed across the substrate, the pump is switched off
The applied coating is dried in an oven at 50° C. for 3 minutes

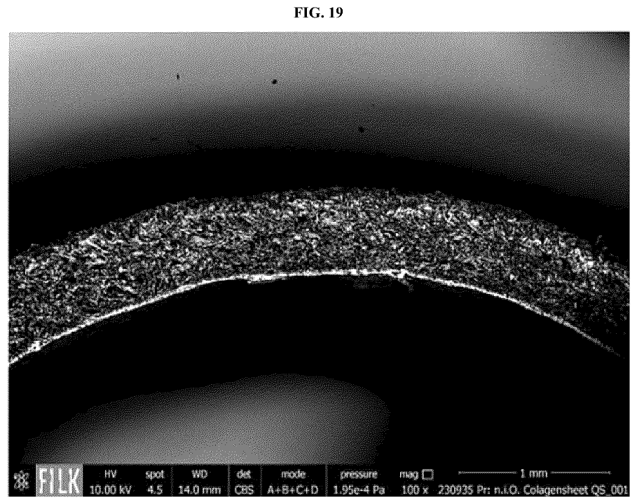

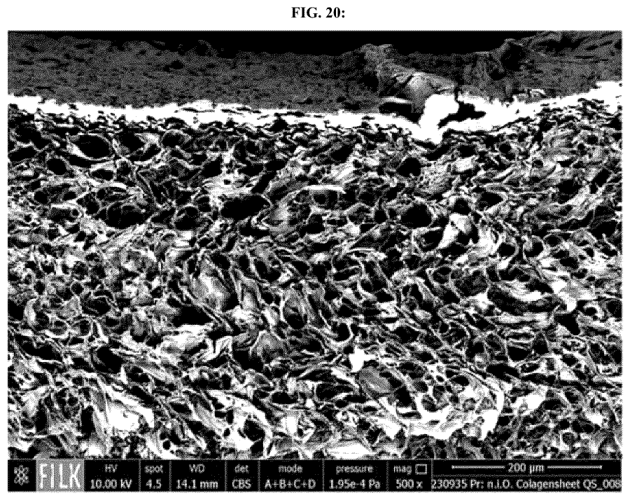

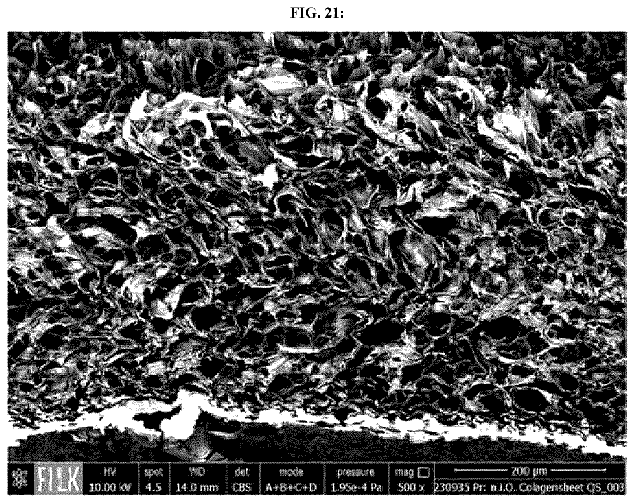

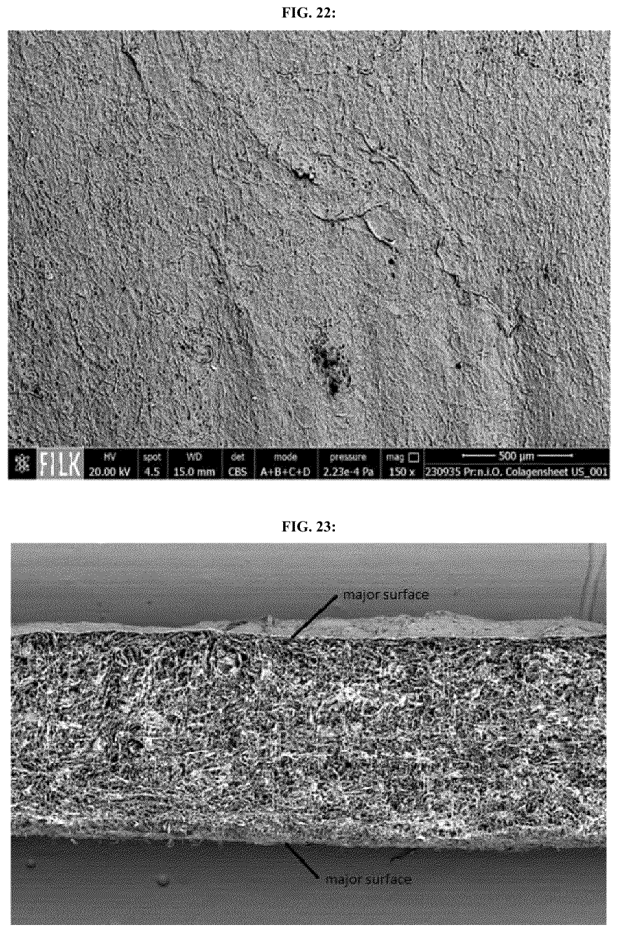

The SEM images of the obtained materials are depicted as FIGS. 18-22. As can be seen, no efficient coating of the porous collagen can be conducted with the coating solution of viscosity of 3 Pa·s. The top view of the coated sample (FIG. 18) looks similarly to the top view of the uncoated material (FIG. 2), which indicates that the coating solution has gone through the pores to the bottom side, which can be also seen in FIG. 22. This results in a substantial collapse of the thickness of the porous substrate as can be seen in FIG. 19. The cross-section views (FIGS. 20 and 21) also indicate that the porous structure has been strongly affected since substantially no cavities and interstices are observed, but only channels. HA from the coating solution can be seen on the both sides of the porous substrate. So, the viscosity of the initial coating solution is a crucial parameter to establish a stable coating film onto the porous material.

1. A method of making a composition comprising a porous material, wherein said porous material is essentially flat and comprises a plurality of open and interconnected pores with pore surfaces, comprising the steps of:
a) Providing a porous material, wherein said porous material is essentially flat and comprises a plurality of open and interconnected pores with pore surfaces, wherein the density of said porous material is in the range of from 0.01 to 1 g/cm³, preferably in the range of from 0.02 to 0.05 g/cm³, most preferred in the range of from 0.02 to 0.04 g/cm³ and wherein said pores have an average diameter in the range of from 10 to 150 μm;
b) Providing a coating solution, wherein the viscosity of said coating solution is in the range of from 5 to 20 Pa·s, preferably from 8 to 15 Pa·s, more preferably from 10 to 14 Pa·s;
c) Providing an applicator means for applying the coating solution onto the surface of said porous material, wherein said applicator means comprises a slot-die;
d) Applying a quantitative amount of the coating solution onto the surface of said porous material to coat said porous material and form a liquid layer by relatively moving said porous material and the applicator means with respect to each other at a speed in the range of from 0.1 to 10 m per minute;
e) Drying said liquid layer.

2. The method of making a composition comprising a porous material according to claim 1, wherein the density of said porous material increases by 10 to 30%, preferably by 10 to 20% after the coating.

3. The method of making a composition comprising a porous material according to claim 1, wherein the quantitative amount of the coating solution applied onto the surface of said porous material in step d) is in the range of from 0.5 to 200 g/m², preferably in the range of from 10 to 100 g/m², most preferred in the range of from 10 to 30 g/m².

4. The method of making a composition comprising a porous material according to claim 1, wherein said coating solution comprises a solvent component and a film forming component.

5. The method of making a composition comprising a porous material according to claim 4, wherein said solvent component is water.

6. The method of making a composition comprising a porous material according to claim 5, wherein the content of water in said coating solution is in the range of from 0.5 to 50 wt %, preferably in the range of from 2 to 35 wt %, more preferably in the range of from 3 to 30 wt %.

7. The method of making a composition comprising a porous material according to claim 4, wherein said film forming component is selected from the group consisting of hyaluronic acid (HA), polyacrylate, polyurethane and polysaccharides, such as alginate, nanocellulose, or carboxymethylcellulose (CMC), or mixtures thereof.

8. The method of making a composition comprising a porous material according to claim 1, wherein said biomaterial is collagen.

9. A composition comprising a porous material obtainable by a method according to claim 1.

10. A composition comprising
a porous substrate comprising a first major surface and a second major surface, wherein said porous substrate is essentially flat and comprises a plurality of open and interconnected pores with pore surfaces extending through the porous substrate from the first major surface to the second major surface, wherein said pores have an average diameter in the range of from 10 to 150 μm;
a coating on the first major surface and/or on the second major surface of said porous substrate, wherein said coating comprises hyaluronic acid (HA), polyacrylate, polyurethane or polysaccharides, such as alginate, nanocellulose, or carboxymethylcellulose (CMC), or mixtures thereof;
characterized in that said composition has water absorption in the range of from 10 to 50 g per g of said composition, preferably from 20 to 40 g per g of said composition.

11. The composition according to claim 10, wherein said coating in the coated porous material is bound to the first major surface and/or on the second major surface of said porous substrate through non-covalent bonds.

12. The composition according to claim 10, wherein the coating on the first major surface and/or on the second major surface of the substrate further comprises water.

13. The composition according to claim 10, wherein the amount of the coating in said composition is in the range of from 0.1 to 10 wt %, preferably from 0.5 to 3 wt % in relation to the weight of said composition.

14. The composition according to claim 10 for use as a medicament.

15. The composition according to claim 10 for use in the treatment of stagnating wounds, split skin graft and ulcers.
